(19) United States
(12) Patent Application Publication
(10) Pub. No.: US 2024/0066051 A1
(43) Pub. Date: Feb. 29, 2024

(54) NASAL SPRAY USING PENTOSAN POLYSULFATE AND MUCOPOLYSACCHARIDE POLYSULFATE FOR COVID-19 PREVENTION AND TREATMENT

(71) Applicant: Rensselaer Polytechnic Institute, Troy, NY (US)

(72) Inventors: Robert John LINHARDT, Albany, NY (US); Fuming ZHANG, Watervliet, NY (US); Jawed FAREED, Westchester, IL (US); Walter JESKE, Woodridge, IL (US)

(73) Assignee: Rensselaer Polytechnic Institute, Troy, NY (US)

(21) Appl. No.: 18/237,624

(22) Filed: Aug. 24, 2023

Related U.S. Application Data
(60) Provisional application No. 63/400,563, filed on Aug. 24, 2022.

(51) Int. Cl. A61K 31/737; A61K 9/00; A61P 31/14

(52) U.S. Cl. CPC A61K 31/737; A61K 9/0043; A61P 31/14

(57) ABSTRACT

Prophylaxis and treatment of patients susceptible to infection by coronaviruses such as severe acute respiratory syndrome coronavirus 2 (SARS-CoV-2) is achieved using compositions including one or more inhibitors including sulfated glycans and/or highly negative charged compounds. The inhibitors bind to wild-type and variant spike glycoproteins (S-proteins or SGPs) of SARS-CoV-2, inhibiting fusion, entry, and infection of a host cell. The inhibitors can include pentosan polysulfate (PPS), mucopolysaccharide polysulfate (MPS), sulfated lactobionic acid, sulodexide, a defibrotide, 4-t-butylcalix[X] arene-p-sulfonic acid, or combinations thereof. The presence of additional sulfo groups in PPS and MPS contribute to the improved inhibitory activity of compositions with those sulfated glycans against COVID-19 compared with compositions containing heparin alone. The compositions can be formulated for nasal delivery to a patient, enabling a simplified treatment regimen effective against an ever increasing number of SARS-CoV-2 variants of concern.

### CROSS REFERENCE TO RELATED APPLICATION(S)

This application claims the benefit of U.S. Provisional Application No. 63/400,563, filed Aug. 24, 2022, which is incorporated by reference as if disclosed herein in its entirety.

### STATEMENT REGARDING FEDERALLY SPONSORED RESEARCH AND DEVELOPMENT

This invention was made with U.S. Government support under Grant Number DMR-1933525 awarded by the National Science Foundation, and Grant Numbers AG069039-01, DK111958, and S10OD0285232 awarded by the National Institutes of Health. The United States Government has certain rights in the invention.

### BACKGROUND

The COVID-19 pandemic, caused by severe acute respiratory syndrome-related coronavirus 2 (SARS-CoV-2), has resulted in a public health disaster and led to millions of deaths globally. Many variants of SARS-CoV-2 have been recognized by the World Health Organization (Alpha, Beta, Gamma, Delta, Omicron, etc.) since the beginning of the pandemic. These variants of concern (VOC) have exhibited increased transmissibility, virulence, and/or a reduced effectiveness of vaccines, resulting in immune breakthrough infections. Up until November 2021, the Delta variant (B.1.617.2) had been the most common COVID-19 variant circulating worldwide since October 2020. With greatly increased transmissibility, however, a new variant, Omicron (B.1.1.529), became the predominant strain.

Glycosaminoglycans (GAGs) are a family of highly negatively charged linear polysaccharides including heparin/heparan sulfate (HS), chondroitin sulfate (CS)/dermatan sulfate (DS), hyaluronan (HA), and keratan sulfate (KS). GAGs interact with various proteins, such as growth factors/receptors, morphogens, chemokines, extracellular matrix proteins, lipoproteins, and pathogens. These interactions play vital roles in pathological processes/diseases such as inflammation, angiogenesis, cancer, neurodegenerative diseases, and infectious diseases. GAG—protein interactions have been targeted for many therapeutic applications.

During the initial stage of host cell invasion, SARS-CoV-2 invades the human host cells through the interaction of its spike glycoproteins (SGPs) with a host cell receptor, angiotensin-converting enzyme 2 (ACE2). In addition, HS on the surface of host cells plays a role as a co-receptor for this viral pathogen—host cell interaction. Without wishing to be bound by theory, HS functions as a cofactor for SARS-CoV-2, binding to ACE2 by interacting with the receptor-binding domain (RBD) at the Si subunit of the SGP, which facilitates the opening of SGP conformation for ACE2 binding.

Although there are several vaccines offering protection for COVID-19, the efficacy of these vaccines for VOCs has been reduced. Supplementing vaccines, some oral or injectable therapeutics have been developed (or repurposed), including remdesivir, favipiravir, simeprevir, various monoclonal antibodies, paxlovid, and molnupiravir, which can inhibit the infection or propagation of SARS-CoV-2. However, the clinical efficacies of many of these agents are highly variable and are most effective only in the first few days of infection. Moreover, apart from monoclonal antibodies, their prophylactic use is not indicated. What is desired, therefore, are effective drugs for both therapeutic and prophylactic uses to combat COVID-19.

### SUMMARY

Aspects of the present disclosure are directed to a composition for inhibiting SARS-CoV-2. In some embodiments, the composition includes one or more inhibitors configured to bind to spike protein (S-protein) of severe acute respiratory syndrome coronavirus 2 (SARS-CoV-2), wherein the one or more inhibitors include pentosan polysulfate, mucopolysaccharide polysulfate, sulfated lactobionic acid, sulodexide, a defibrotide, 4-t-butylcalix[X] arene-p-sulfonic acid, or combinations thereof. In some embodiments, X is 6 or 8. In some embodiments, the 4-t-butylcalix[X] arene-p-sulfonic acid includes GL-2179, GL-2021, GL-2029, GL-288-Y-1, GL-522-Y-1, GL-522Y-1 Calcium, or combinations thereof. In some embodiments, the one or more inhibitors include pentosan polysulfate, mucopolysaccharide polysulfate, or combinations thereof. In some embodiments, the one or more inhibitors have a molecular weight greater than about 5 kDa. In some embodiments, the one or more inhibitors have a molecular weight greater than about 10 kDa. In some embodiments, the composition includes one or more additional active ingredients. In some embodiments, the composition includes pharmaceutically acceptable adjuvants, diluents, excipients, carriers, or combinations thereof. In some embodiments, the composition is formulated for nasal delivery.

Aspects of the present disclosure are directed to a method of treating an individual with SARS-CoV-2 including identifying a SARS-CoV-2 infection in a patient and nasally administering an effective amount of a composition to the patient, the composition including one or more inhibitors configured to bind to S-protein of SARS-CoV-2, wherein the composition is formulated for nasal delivery. In some embodiments, nasally administering to the patient the effective amount of the composition produces a peak plasma concentration in the patient less than about 0.5 μg/mL.

As discussed above, in some embodiments, the one or more inhibitors include pentosan polysulfate, mucopolysaccharide polysulfate, sulfated lactobionic acid, sulodexide, a defibrotide, GL-2179, GL-2021, GL-2029, GL-288-Y-1, GL-522-Y-1, GL-522Y-1 Calcium, or combinations thereof. In some embodiments, the one or more inhibitors include pentosan polysulfate, mucopolysaccharide polysulfate, or combinations thereof. In some embodiments, the one or more inhibitors have a molecular weight greater than about 5 kDa. In some embodiments, the one or more inhibitors have a molecular weight greater than about 10 kDa. In some embodiments, the composition includes pharmaceutically acceptable adjuvants, diluents, excipients, carriers, or combinations thereof. In some embodiments, the composition is formulated for nasal delivery.

Aspects of the present disclosure are directed to a method of treating or preventing an infection caused by SARS-CoV-2 in a patient including administering an effective amount of a composition to a patient susceptible to infection by SARS-CoV-2, the composition including one or more inhibitors configured to bind to spike protein (S-protein) of SARS-CoV-2, wherein the one or more inhibitors include one or more sulfated glycans having a plurality of repeating disaccharide subunits, wherein the disaccharide subunits include 3 or more sulfo groups. In some embodiments, administering to the patient the effective amount of the composition produces a peak plasma concentration in the patient less than about 0.5 μg/mL. In some embodiments, administering to the patient the effective amount of the composition includes administering the composition nasally, wherein the composition is formulated for nasal delivery.

As discussed above, in some embodiments, the sulfated glycans include pentosan polysulfate, mucopolysaccharide polysulfate, or combinations thereof. In some embodiments, the sulfated glycans have a molecular weight greater than about 5 kDa. In some embodiments, the sulfated glycans have a molecular weight greater than about 10 kDa. In some embodiments, the composition includes one or more additional active ingredients. In some embodiments, the composition includes pharmaceutically acceptable adjuvants, diluents, excipients, carriers, or combinations thereof. In some embodiments, the composition is formulated for nasal delivery.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 portrays graphs showing binding by pseudotyped severe acute respiratory syndrome coronavirus 2 (SARS-CoV-2) viral particles to surface-immobilized heparin in the presence of pentosan polysulfate (PPS) and mucopolysaccharide polysulfate (MPS), consistent with embodiments of the present disclosure;

FIG. 2 portrays results showing inhibition of pseudotyped wild-type and variant SARS-CoV-2 virus in the presence of PPS and MPS, consistent with embodiments of the present disclosure;

FIG. 3 portrays a plurality of chemical structures of SARS-CoV-2 spike protein inhibitors for inclusion in compositions according to embodiments of the present disclosure;

FIG. 4 portrays surface plasmon resonance (SPR) sensorgrams of wild-type and variant SARS-CoV-2 spike proteins with heparin;

FIG. 5 is a graph showing normalized binding of SARS-CoV-2 spike protein to surface immobilized heparin by inhibitors for inclusion in compositions according to embodiments of the present disclosure;

FIGS. 6A-6F portray graphs showing IC₅₀ measurements of wild-type SARS-CoV-2 spike protein binding to heparin in the presence of various inhibitors, including heparin (FIGS. 6A-6B); PPS (FIGS. 6C-6D); and MPS (FIGS. 6E-6F);

FIG. 7 portrays graphs showing of normalized binding by variant SARS-CoV-2 spike proteins to surface-immobilized heparin in the presence of PPS and MPS, consistent with embodiments of the present disclosure;

FIG. 8 is a chart of a method treating an individual with a coronavirus, e.g., SARS-CoV-2 according to some embodiments of the present disclosure; and

FIG. 9 is a chart of a method of treating or preventing an infection caused by SARS-CoV-2 in a patient according to some embodiments of the present disclosure.

### DETAILED DESCRIPTION

Some embodiments of the present disclosure are directed to a composition for inhibiting coronaviruses, e.g., severe acute respiratory syndrome coronavirus 2 (SARS-CoV-2), common cold coronaviruses, etc., or combinations thereof. In some embodiments, the composition is configured to bind to spike glycoproteins, also referred to as “spike proteins,” “5-proteins,” or “SGPs,” at the surface of the viral particles, e.g., SARS-CoV-2 spike proteins. In some embodiments, the composition includes one or more inhibitors configured to bind to spike glycoproteins. In some embodiments, the composition includes one or more naturally-occurring spike glycoprotein inhibitors, one or more modified naturally-occurring spike glycoprotein inhibitors, one or more synthetic spike glycoprotein inhibitors, or combinations thereof. In some embodiments, the composition includes one or more inhibitors configured to bind to SARS-CoV-2 spike protein. In some embodiments, the composition includes one or more inhibitors configured to bind to wild-type (WT) SARS-CoV-2 spike protein. In some embodiments, the composition includes one or more inhibitors configured to bind to one or more variant SARS-CoV-2 spike proteins, such as Beta, Gamma, Delta, e.g., B.1.617.2, Omicron, e.g., B.1.1.529, or combinations thereof. In some embodiments, the composition includes a plurality, e.g., two or more, inhibitors configured to bind to SGPs. In some embodiments, the composition includes a plurality, e.g., two or more, inhibitors configured to bind to SARS-CoV-2 spike proteins.

In some embodiments, the composition is formulated to deliver the one or more inhibitors to the site of an active coronavirus infection, e.g., by SARS-CoV-2. In some embodiments, the composition is formulated to deliver the one or more inhibitors to the initial site of a coronavirus infection. In some embodiments, the composition is formulated for prophylactic delivery via any suitable route. In some embodiments, the composition is included in a therapeutic for administration to a patient, e.g., orally, nasally, via inhalation, nebulization, transdermally, intravenously, or combinations thereof. In some embodiments, the composition is formulated for nasal delivery. In some embodiments, the nasal composition is configured for prophylactic delivery against coronaviruses, e.g., SARS-CoV-2, to a patient's nasal passages.

In some embodiments, the one or more inhibitors include one or more highly charged compounds. In some embodiments, the one or more inhibitors includes one or more highly negatively charged compounds, pharmaceutically acceptable salts thereof, or combinations thereof. In some embodiments, the one or more highly charged compounds include one or more modifications configured to increase binding affinity for SGPs, binding affinity for SARS-CoV-2 SGPs, pharmaceutical acceptability, etc., or combinations, e.g., addition and/or substitution of one or more functional groups on the highly charged compound. In some embodiments, the one or more inhibitors include a defibrotide. In some embodiments, the one or more inhibitors include a 4-t-butylcalix[X] arene-p-sulfonic acid. In some embodiments, X is at least 6. In some embodiments, X is 6. In some embodiments, X is 8. In some embodiments, the one or more inhibitors include GL-2179, GL-2021, GL-2029, GL-288-Y-1, GL-522-Y-1, GL-522Y-1 Calcium, or combinations thereof. In some embodiments, the one or more highly charged compounds have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

In some embodiments, the one or more inhibitors include one or more sulfated polysaccharides or glycans, pharmaceutically acceptable salts thereof, or combinations thereof. In some embodiments, the sulfated glycans include a plurality of repeating disaccharide subunits. In some embodiments, the inhibitors include one or more glycosaminoglycans, also referred to herein as “GAGs.” In some embodiments, the disaccharide subunits include an amount of sulfo groups thereon. In some embodiments, the disaccharide subunits include 3 or more sulfo groups. In some embodiments, the disaccharide subunits include 4 or more sulfo groups. In some embodiments, the one or more sulfated glycans include one or more modifications configured to increase binding affinity for SGPs, binding affinity for SARS-CoV-2 SGPs, pharmaceutical acceptability, etc., or combinations thereof, e.g., addition and/or substitution of one or more functional groups on the sulfated glycan. In some embodiments, the one or more inhibitors include pentosan polysulfate (PPS), mucopolysaccharide polysulfate (MPS), sulfated lactobionic acid, pharmaceutically acceptable salts thereof, or combinations thereof. In some embodiments, the one or more inhibitors include a mixture of heparin and at least one other sulfated polysaccharide, e.g., dermatan sulfate. In some embodiments, the one or more inhibitors include sulodexide. In some embodiments, the one or more sulfated glycans have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

In some embodiments, the composition includes one or more highly charged compounds and one or more sulfated glycans. In some embodiments, the composition includes one or more additional active ingredients, one or more pharmaceutically acceptable adjuvants, diluents, excipients, carriers, or combinations thereof.

In some embodiments, the composition is included in a nutraceutical. In some embodiments, the nutraceutical includes inhibitors derived from GRAS organisms (Generally Recognized as Safe). In some embodiments, the composition is included in a coating, e.g., a coating layer for face masks or other surfaces, tightly binding the virus and improving the effectiveness of the surface to inhibit infection, e.g., enhance the effectiveness of the mask to block transmission of the virus. In some embodiments, the composition is included in filter materials, e.g., in A/C and other HVAC systems in buildings.

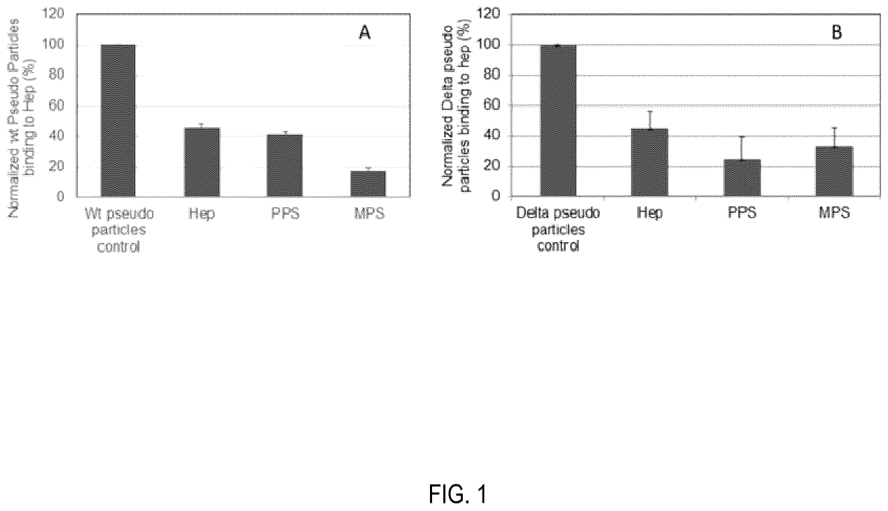

Referring now to FIG. 1, solution/surface competition experiments were carried out to test the inhibition of pseudovirus particle (wild-type and Delta variant)—heparin interaction by PPS and MPS. PPS, a heparin mimetic with a highly sulfated polysaccharide backbone, is synthesized through the chemical sulfonation of a plant-derived β-(1→4)-xylan. PPS is an FDA-approved active pharmaceutical ingredient of the oral drug Elmiron™. The FDA has approved PPS as an oral anti-thrombotic agent for the management of patients with interstitial cystitis, and it is also used for clinical disorders such as antagonism of enzymatic activities and inhibition of HIV infectivity. MPS is a semisynthetic GAG with a backbone that is isolated from mammalian cartilage before its chemical sulfation. MPS has been used for the topical treatment of superficial phlebitis, hematomas, and sports-related injuries. Both PPS and MPS showed stronger inhibition of both wild-type and Delta variant pseudotyped viral particle binding to heparin surface compared with heparin control.

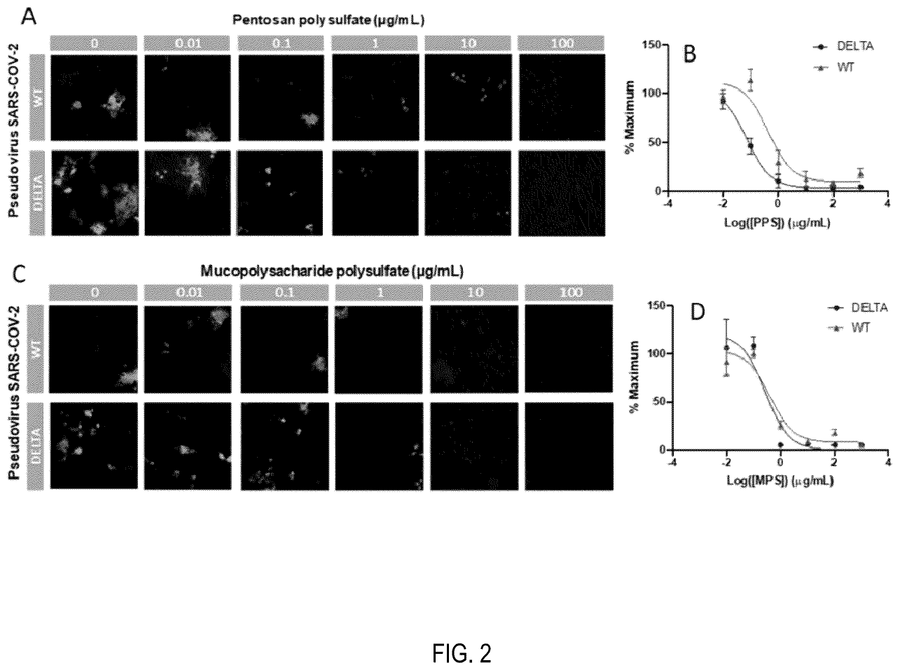

Referring now to FIG. 2, an in vitro SARS-CoV-2 pseudotyped viral particle neutralization assay was performed. Sulfated glycans such as heparin have been shown to inhibit viral infection by interacting with the SARS-CoV-2 spike protein. Without wishing to be bound by theory, upon initially contacting the surface of a host cell, SARS-CoV exploit host cell proteases to prime their surface SGPs for fusion activation, which is achieved by receptor binding, low pH, or both. Activated SGP undergoes a conformational change followed by an initiated fusion reaction with the host cell membrane. Endocytosed virions are further processed by the endosomal protease cathepsin L in the late endosome. Additionally, receptors involved in fusion activation of SARS-CoV include heparan sulfate (HS) and angiotensin-converting enzyme 2 (ACE2). Without wishing to be bound by theory, GAGs contribute to SARS-CoV-2 fusion activation and host cell entry via spike protein binding. Sulfated polysaccharides bind tightly to the spike protein of SARS-CoV-2, and thus, they can act as decoys to interfere with spike protein binding to the HS co-receptor in host tissues, inhibiting viral infection. Competitive inhibition by a concentration of highly charged extracellular compounds such as sulfated glycans can be used to prevent SGP binding to host cells and thus prevent cell fusion, entry, and cellular infection.

It has been shown that heparin interacts with the SARS-CoV-2 S-protein with high avidity. It has been proposed that HS facilitates host cell entry of SARS-CoV-2 as a co-receptor of ACE2. Without wishing to be bound by theory, protein binding to heparin/HS results from the ionic- or hydrogen-bonding interactions of basic amino acid residues, placed in defined motifs within the protein, with the anionic carboxyl and sulfo groups within these GAGs. With the greater prevalence of new SARS-CoV-2 variants of concern (VOC), additional S-protein mutants have been identified. As the primary antigen of SARS-CoV-2, mutations of the S-protein greatly alter the viral infectivity, disease severity, and effectiveness of vaccines.

Thus, the ability of PPS and MPS to inhibit viral particle entry was investigated using a cell-based neutralization assay. HEK293T cells were used that stably expressed the ACE2 receptor (HEK293T-ACE2). Six different concentrations were tested at 1:10 viral dilution, thus enabling the determination of an IC₅₀ value for viral inhibition based on the expression of EGFP as a marker for functional viral entry. Briefly, PPS or MPS was incubated with pseudovirus particles for 1 hour at 37° C., after which the mixture was added to the HEK293T-ACE2 cells and incubated for 4 hours. These incubation steps were performed under serum-free conditions as, without wishing to be bound by theory, sera often include growth factors that interact with polysaccharides, thus interfering with its interaction with the spike protein. After the 4 hour incubation, there was a medium exchange with serum to sustain cell growth for 48 hours, after which the plates were assayed for expression of EGFP. For each concentration, the percentage of infected cells was normalized to the percentage of infected cells relative to the control (no PPS and MPS and 1:10 viral dilution). The lowest dilution (PPS and MPS concentration of 1 μg/mL) provided >80% inhibition of viral particle entry for both the WT and Delta variant. The IC₅₀ values (concentration of competing analyte resulting in a 50% decrease in response units (RU)) of PPS for the WT and Delta variant were 0.45 and 0.07 μg/mL, and the IC₅₀ values of MPS for the WT and Delta variant were 0.42 and 0.28 μg/mL, respectively.

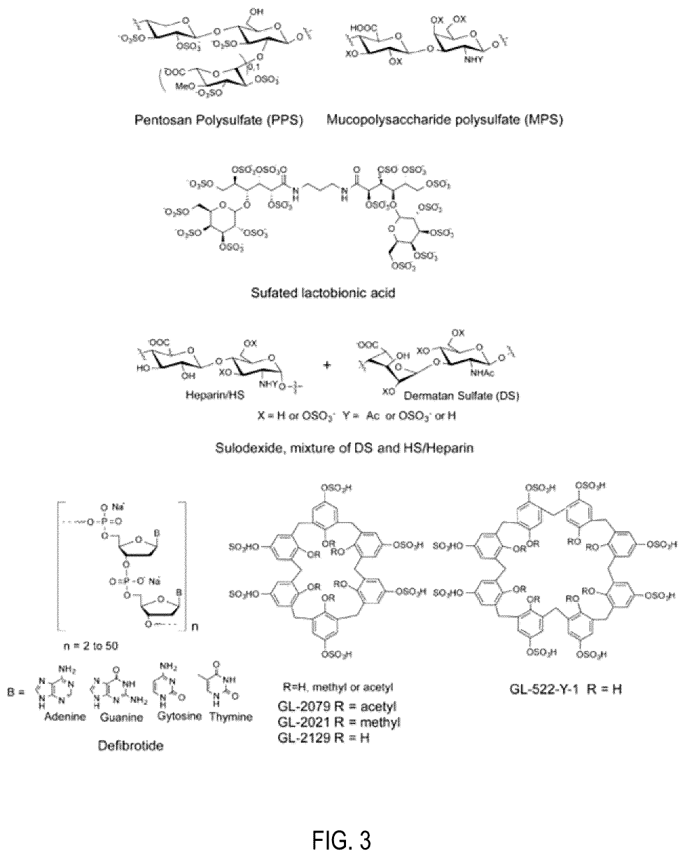

Referring now to FIG. 3, some inhibitors consistent with embodiments of the present disclosure are shown. In an exemplary embodiments, molecular interactions of sulfated glycans, e.g., PPS (MW 6,500) and MPS (MW 14, 500) and SARS-CoV-2 S-Protein were analyzed using surface plasmon resonance (SPR).

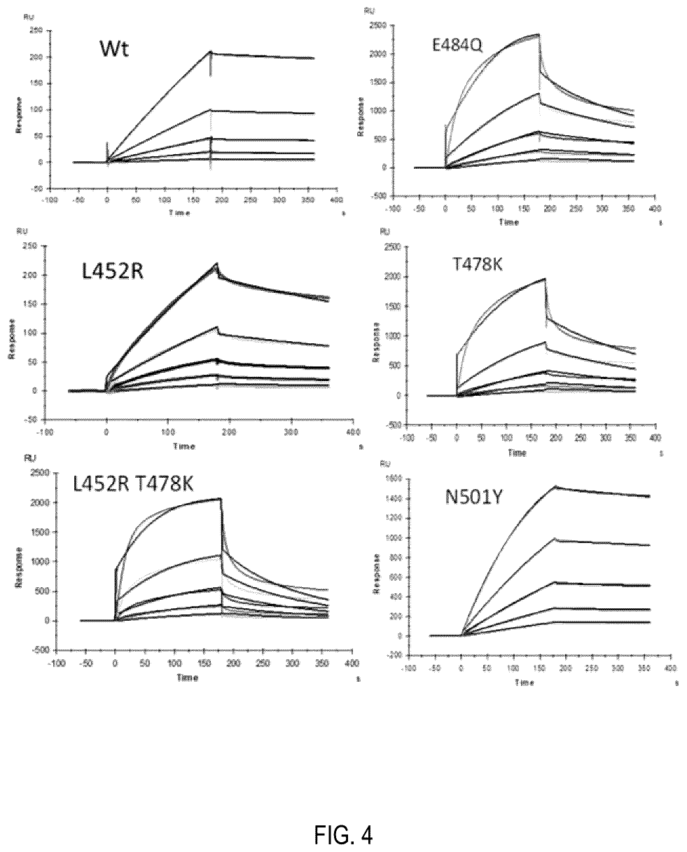

Referring now to FIG. 4, SPR was applied to measure the binding kinetics and affinity of SARS-CoV-2 S-protein receptor-binding domain (RBD) (WT and different VOC) interaction with heparin using a sensor chip with immobilized heparin. The resulting sensorgrams were used to determine binding kinetics and affinity, i.e., association rate constant: kₐ; dissociation rate constant: k_{d}; and binding equilibrium dissociation constant: K_{D} (K_{D}=k_{d}/kₐ) by globally fitting the entire association and dissociation phases using the 1:1 Langmuir binding model from T200 Evaluation software. Binding kinetic parameters (kₐ and k_{d}) and affinity (K_{D}) were calculated (see Table 1) from sensorgrams and globally fitted to the 1:1 Langmuir model from T200 Evaluation software. The binding kinetics and affinities of the different VOC S-protein RBDs were comparable to the WT version except for N501Y, which showed higher affinity to heparin, and L452R, which showed lower affinity to heparin.

TABLE 1
Summary of kinetic data of heparin and SARS-CoV-2 S-protein RBD (WT
and VOC) interactions. The data with (±) in parentheses represent
standard deviations (SD) from the global fitting of five injections.

| Interaction | kₐ (1/MS) | | k_{d} (1/S) | | K_{D} (M) |
|---|---|---|---|---|---|
| SARS-CoV-2 S- | 1427 | (±26) | 2.5 × 10⁻⁴ | (±2.7 × 10⁻⁶) | 1.8 × 10⁻⁷ |
| protein RBD WT | | | | | |
| SARS-CoV-2 S- | 5.2 × 10⁴ | (±1.6 × 10³) | 0.011 | (±2.9 × 10⁻⁴) | 2.0 × 10⁻⁷ |
| protein RBD E484Q | | | | | |
| SARS-CoV-2 S- | 2.3 × 10⁴ | (±100) | 0.014 | (±5.6 × 10⁻⁴) | 5.9 × 10⁻⁷ |
| protein RBD | | | | | |
| L452R + T478K | | | | | |
| SARS-CoV-2 S- | 1.2 × 10⁴ | (±100) | 6.0 × 10⁻³ | (±7.3 × 10⁻⁵) | 4.9 × 10⁻⁷ |
| protein RBD T478K | | | | | |
| SARS-CoV-2 S- | 161 | (±3.3) | 1.3 × 10⁻³ | (±5.4 × 10⁻⁶) | 8.4 × 10⁻⁶ |
| protein RBD L452R | | | | | |
| SARS-CoV-2 S- | 1.7 × 10⁴ | (±81) | 4.2 × 10⁻⁴ | (±2.0 × 10⁻⁶) | 2.5 × 10⁻⁸ |

protein RBD N501Y

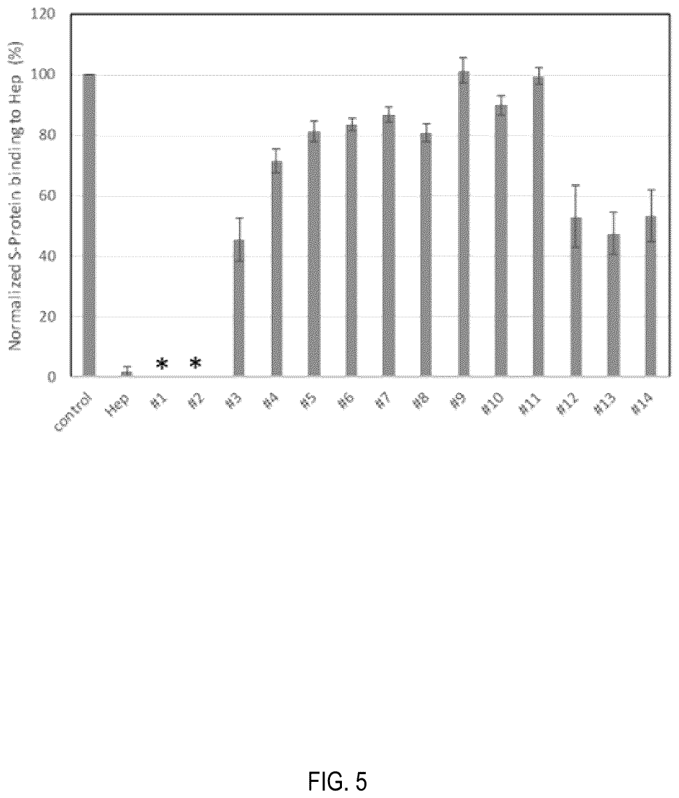

Referring now to FIG. 5, solution/surface competition experiments were performed using SPR to examine the inhibition of inhibitors such as sulfated glycans and additional highly negatively charged compounds to the interaction between heparin (on surface) with S-protein. PPS and MPS potently inhibited the S-protein—heparin interaction, while sulfated lactobionic acid, GL-288-Y-1, GL-522-Y-1, and GL-522Y-1 calcium exhibited a more modest inhibition activity. Relatively lower inhibition activity of different versions of defibrotide was observed. Based on these data, PPS and MPS were selected for further investigation.

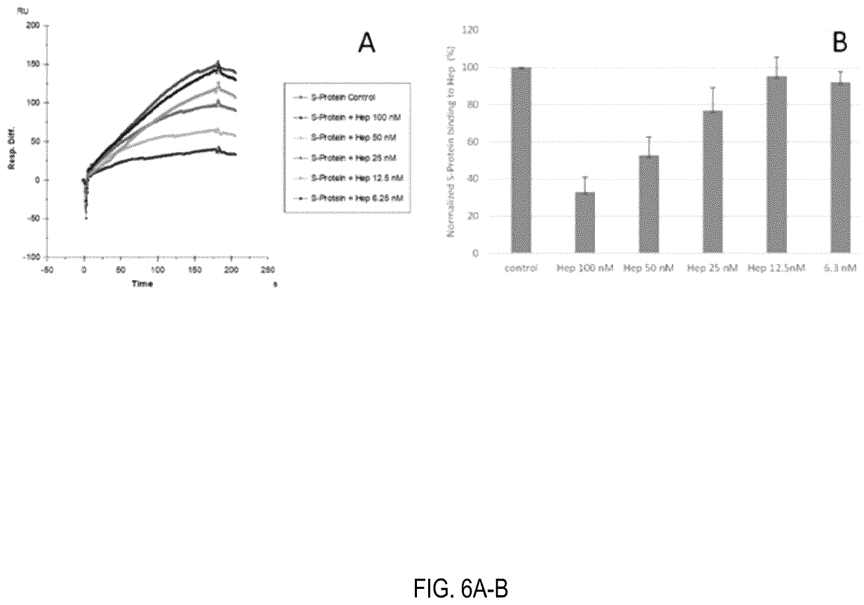

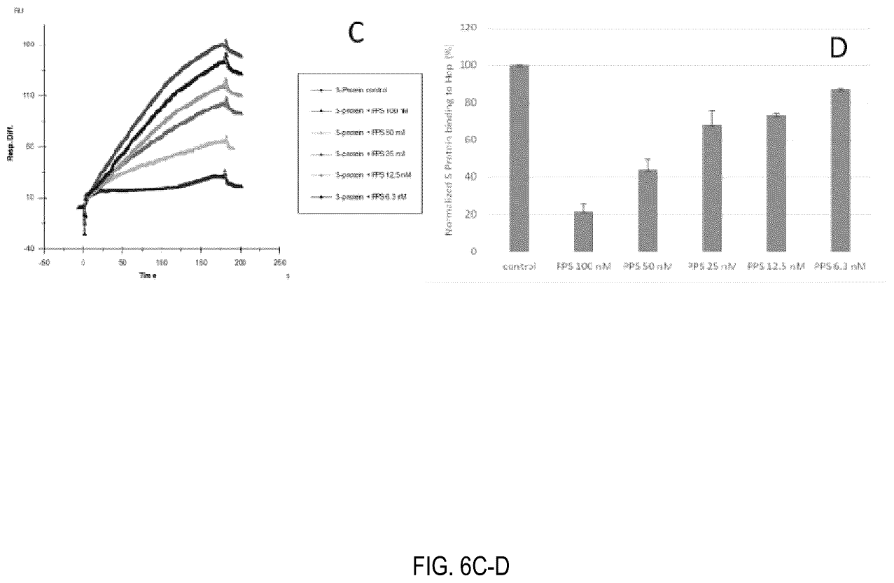

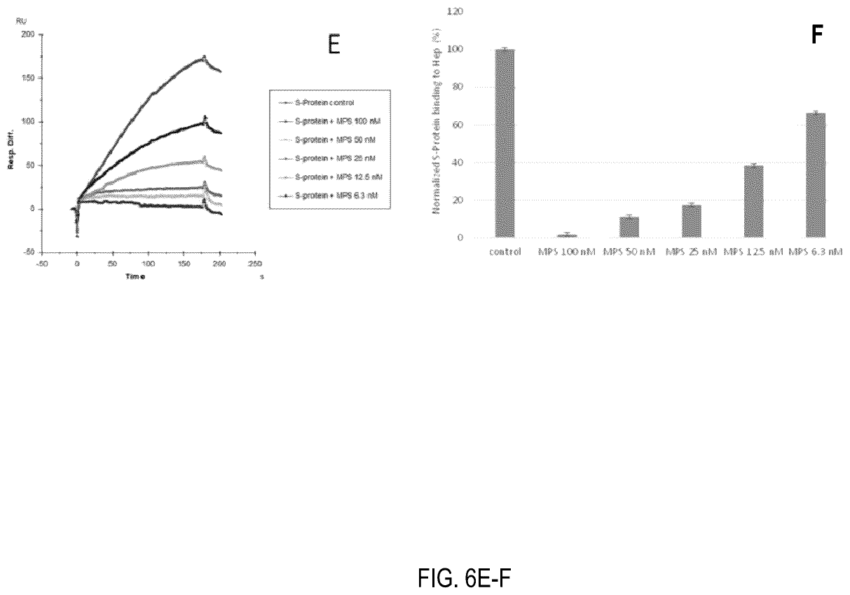

Referring now to FIGS. 6A-6F, solution competition dose—response analysis between surface-immobilized heparin and soluble PPS and MPS was performed to calculate IC₅₀ values and to quantify the inhibition by PPS and MPS of the interaction between heparin (on surface) and S-protein RBD (WT). S-protein RBD was pre-mixed with different concentrations of PPS, MPS, or heparin before injection into the heparin chip. When the active binding sites on the S-protein RBD were occupied by glycan in solution, its binding to the surface-immobilized heparin decreased, resulting in a reduction in signal in a concentration-dependent fashion. The IC₅₀ values were calculated from the plots S-protein RBD binding signal (normalized) versus glycan concentration in solution. The competitive binding studies revealed that the IC₅₀ of PPS and MPS against the S-protein RBD binding to immobilized heparin was ˜35 nM and ˜9 nM, respectively, which was much lower than the IC₅₀ for heparin (IC₅₀=56 nM). Without wishing to be bound by theory, this could be due to the level of sulfation being higher for MPS and PPS compared with heparin. The average heparin disaccharide contains ˜2.7 sulfo groups, while MPS disaccharide has >4 sulfo groups and PPS disaccharide has >3 sulfo groups. By way of example, the 2,3-disulfated polyxylan oligosaccharide is a sugar moiety contributing to PPS' binding to the S-protein RBD, MPS has a heparin-like molecular structure with a high level of sulfo groups, which could allow this glycan to interact with the S-protein, etc.

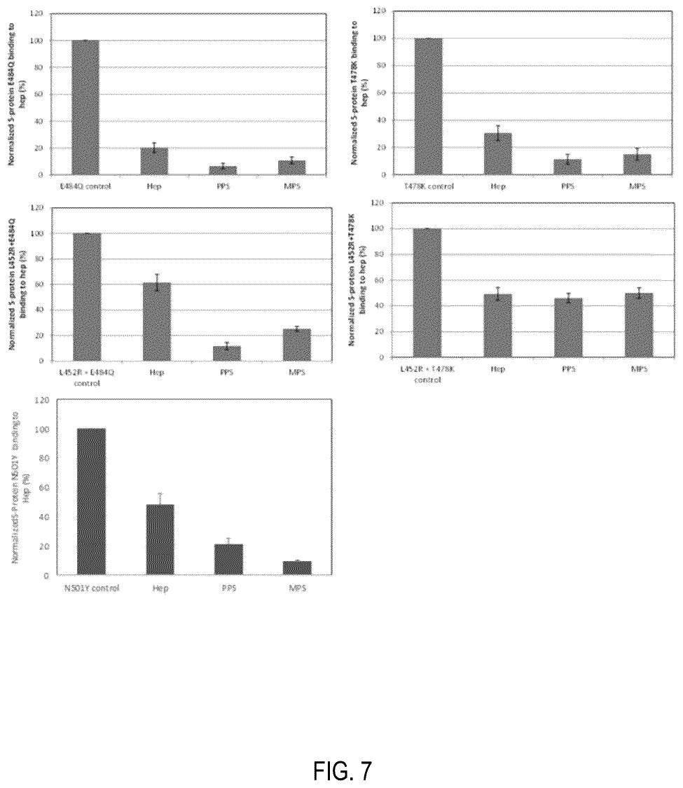

Referring now to FIG. 7, solution/surface competition experiments were also performed using SPR to examine the inhibition of different S-protein RBD VOC—heparin interactions by PPS and MPS. Using the same concentration of PPS, MPS, and heparin (5 ng/mL), PPS and MPS showed stronger inhibition of most of S-protein RBD mutants tested than heparin, with the exception of the L452R+T478K mutant, which showed comparable inhibition to heparin.

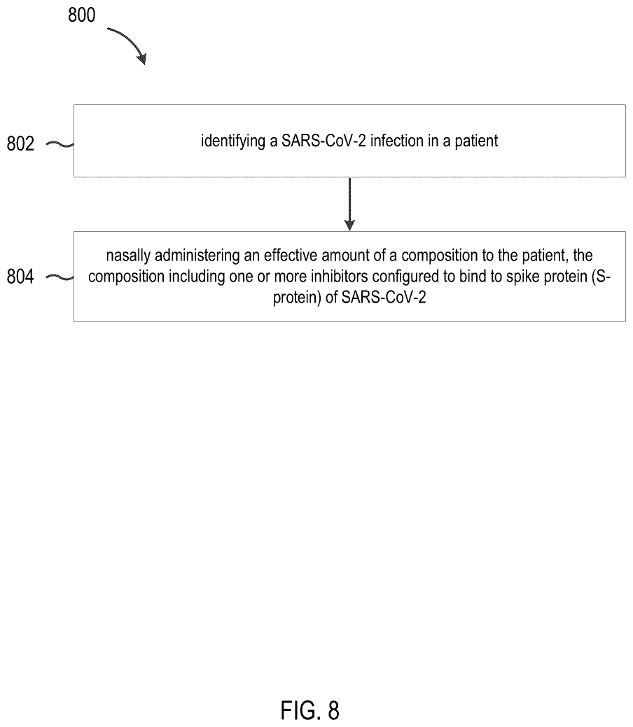

Referring now to FIG. 8, some embodiments of the present disclosure are directed to a method 800 of treating an individual with a coronavirus, e.g., SARS-CoV-2. At 802, a coronavirus infection in a patient such as SARS-CoV-2 is identified. At 804, an effective amount of a composition is nasally administered to the patient. In some embodiments, nasally administering 804 to the patient the effective amount of the composition produces a peak plasma concentration in the patient less than about 0.5 μg/mL. In some embodiments, nasally administering 804 to the patient the effective amount of the composition produces a peak plasma concentration in the patient less than about 0.3 μg/mL. In some embodiments, nasally administering 804 to the patient the effective amount of the composition produces a peak plasma concentration in the patient less than about 0.1 μg/mL.

As discussed above, in some embodiments, the composition is formulated for nasal delivery. In some embodiments, the nasal composition is configured for prophylactic delivery against coronaviruses, e.g., SARS-CoV-2, to a patient's nasal passages. In some embodiments, the composition is configured to bind to SGPs at the surface of viral particles such as SARS-CoV-2. In some embodiments, the composition includes one or more inhibitors configured to bind to SGPs. In some embodiments, the composition includes one or more inhibitors configured to bind to SARS-CoV-2 spike protein. In some embodiments, the composition includes a plurality, e.g., two or more, inhibitors configured to bind to SARS-CoV-2 spike protein.

In some embodiments, the one or more inhibitors include one or more highly charged compounds. In some embodiments, the one or more inhibitors includes one or more highly negatively charged compounds, pharmaceutically acceptable salts thereof, or combinations thereof. In some embodiments, the one or more highly charged compounds include one or more modifications configured to increase binding affinity for SGPs, binding affinity for SARS-CoV-2 SGPs, pharmaceutical acceptability, etc., or combinations, e.g., addition and/or substitution of one or more functional groups on the highly charged compound. In some embodiments, the one or more inhibitors include a defibrotide. In some embodiments, the one or more inhibitors include a 4-t-butylcalix[X] arene-p-sulfonic acid. In some embodiments, X is at least 6. In some embodiments, X is 6. In some embodiments, X is 8. In some embodiments, the one or more inhibitors include GL-2179, GL-2021, GL-2029, GL-288-Y-1, GL-522-Y-1, GL-522Y-1 Calcium, or combinations thereof. In some embodiments, the one or more highly charged compounds have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

In some embodiments, the one or more inhibitors include one or more sulfated polysaccharides or glycans, pharmaceutically acceptable salts thereof, or combinations thereof. In some embodiments, the sulfated glycans include a plurality of repeating disaccharide subunits. In some embodiments, the inhibitors include one or more GAGs. In some embodiments, the disaccharide subunits include an amount of sulfo groups thereon. In some embodiments, the disaccharide subunits include 3 or more sulfo groups. In some embodiments, the disaccharide subunits include 4 or more sulfo groups. In some embodiments, the one or more sulfated glycans include one or more modifications configured to increase binding affinity for SGPs, binding affinity for SARS-CoV-2 SGPs, pharmaceutical acceptability, etc., or combinations, e.g., addition and/or substitution of one or more functional groups on the sulfated glycan. In some embodiments, the one or more inhibitors PPS, MPS, sulfated lactobionic acid, pharmaceutically acceptable salts thereof, or combinations thereof. In some embodiments, the one or more inhibitors include a mixture of heparin and at least one other sulfated polysaccharide, e.g., dermatan sulfate. In some embodiments, the one or more inhibitors include sulodexide. In some embodiments, the one or more sulfated glycans have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

As discussed above, in some embodiments, the composition includes one or more highly charged compounds and one or more sulfated glycans. In some embodiments, the composition includes one or more additional active ingredients, one or more pharmaceutically acceptable adjuvants, diluents, excipients, carriers, or combinations thereof.

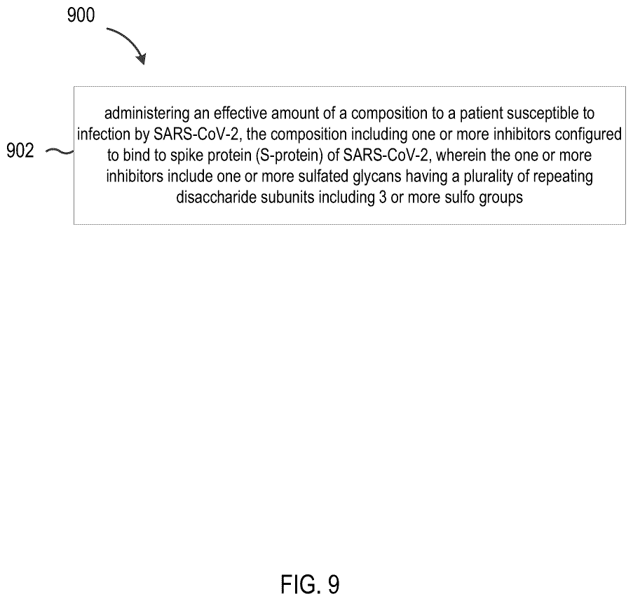

Referring now to FIG. 9, some embodiments of the present disclosure are directed to a method 900 of treating or preventing an infection caused by a coronavirus, e.g., SARS-CoV-2, in a patient. In some embodiments, at 902, an effective amount of a composition is administered to a patient susceptible to infection by the coronavirus. In some embodiments, administering 902 to the patient the effective amount of the composition produces a peak plasma concentration in the patient less than about 0.5 μg/mL. In some embodiments, administering 902 to the patient the effective amount of the composition produces a peak plasma concentration in the patient less than about 0.3 μg/mL. In some embodiments, administering 902 to the patient the effective amount of the composition produces a peak plasma concentration in the patient less than about 0.1 μg/mL. In some embodiments, the composition is administered to a patient orally, nasally, via inhalation, nebulization, transdermally, intravenously, or combinations thereof. In some embodiments, the composition is formulated for nasal delivery. In some embodiments, the nasal composition is configured for prophylactic delivery against the coronavirus, e.g., SARS-CoV-2, to a patient's nasal passages.

As discussed above, in some embodiments, the composition is configured to bind to SGPs at the surface of viral particles such as SARS-CoV-2. In some embodiments, the composition includes one or more inhibitors configured to bind to SGPs. In some embodiments, the composition includes one or more inhibitors configured to bind to SARS-CoV-2 spike protein. In some embodiments, the composition includes a plurality, e.g., two or more, inhibitors configured to bind to SARS-CoV-2 spike protein.

In some embodiments, the one or more inhibitors include one or more highly charged compounds. In some embodiments, the one or more inhibitors includes one or more highly negatively charged compounds, pharmaceutically acceptable salts thereof, or combinations thereof. In some embodiments, the one or more highly charged compounds include one or more modifications configured to increase binding affinity for SGPs, binding affinity for SARS-CoV-2 SGPs, pharmaceutical acceptability, etc., or combinations, e.g., addition and/or substitution of one or more functional groups on the highly charged compound. In some embodiments, the one or more inhibitors include a defibrotide. In some embodiments, the one or more inhibitors include a 4-t-butylcalix[X] arene-p-sulfonic acid. In some embodiments, X is at least 6. In some embodiments, X is 6. In some embodiments, X is 8. In some embodiments, the one or more inhibitors include GL-2179, GL-2021, GL-2029, GL-288-Y-1, GL-522-Y-1, GL-522Y-1 Calcium, or combinations thereof. In some embodiments, the one or more highly charged compounds have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

In some embodiments, the one or more inhibitors include one or more sulfated polysaccharides or glycans, pharmaceutically acceptable salts thereof, or combinations thereof. In some embodiments, the sulfated glycans include a plurality of repeating disaccharide subunits. In some embodiments, the inhibitors include one or more GAGs. In some embodiments, the disaccharide subunits include an amount of sulfo groups thereon. In some embodiments, the disaccharide subunits include 3 or more sulfo groups. In some embodiments, the disaccharide subunits include 4 or more sulfo groups. In some embodiments, the one or more sulfated glycans include one or more modifications configured to increase binding affinity for SGPs, binding affinity for SARS-CoV-2 SGPs, pharmaceutical acceptability, etc., or combinations, e.g., addition and/or substitution of one or more functional groups on the sulfated glycan. In some embodiments, the one or more inhibitors PPS, MPS, sulfated lactobionic acid, pharmaceutically acceptable salts thereof, or combinations thereof. In some embodiments, the one or more inhibitors include a mixture of heparin and at least one other sulfated polysaccharide, e.g., dermatan sulfate. In some embodiments, the one or more inhibitors include sulodexide. In some embodiments, the one or more sulfated glycans have a molecular weight greater than about 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, or combinations thereof.

As discussed above, in some embodiments, the composition includes one or more highly charged compounds and one or more sulfated glycans. In some embodiments, the composition includes one or more additional active ingredients, one or more pharmaceutically acceptable adjuvants, diluents, excipients, carriers, or combinations thereof.

### EXAMPLES

Fourteen compounds were collected from their manufacturers. SARS-CoV-2 S-protein RBD wild type (WT) and N501Y were expressed in Expi293F cells. SARS-CoV-2 S-protein RBD mutants (related to Delta variants of SARS-CoV-2) were purchased from Sino Biological US Inc. (Wayne, PA, USA). SARS-CoV-2 pseudoviral particles (WT and Delta variant) were prepared. Sensor SA chips were from Cytiva (Uppsala, Sweden). SPR measurements were performed on a BIAcore 3000 operated using BIAcore 3000 or T200 SPR (Cytiva, Uppsala, Sweden). The cell line HEK293T was purchased from American Type Culture Collection (ATCC, Manassas, VA, USA).

SPR Measurements of Interactions between Heparin and S-Proteins. Preparation of heparin SPR chip: Heparin (2 mg) and amine-PEG3-Biotin (2 mg, Pierce, Rockford, IL, USA) were dissolved in 200 μL H₂O; 10 mg NaCNBH₃ was added. The reaction mixture was heated at 70° C. for another 24 hours; after that, a further 10 mg NaCNBH₃ was added, and the reaction was heated at 70° C. for another 24 hours. The mixture was desalted using a spin column (3000 MWCO). Biotinylated heparin was freeze-dried for heparin chip preparation. In brief, a 20 μL solution of biotinylated heparin (0.1 mg/mL) in HBS-EP+ buffer (0.01 M 4-(2-hydroxyethyl)-1-piperazineethanesulfonic acid, 0.15 M NaCl, 3 mM ethylenediaminetetraacetic acid, 0.05% surfactant P20, pH 7.4) was injected over flow cell 2 (FC2) of the SA chip at a flow rate of 10 μL/min. The successful immobilization of heparin was confirmed by the observation of a ˜200 resonance unit (RU) increase in the sensor chip. The control flow cell (FC1) was prepared by 1 minute injection with saturated biotin.

Binding kinetics and affinity measurement: Different dilutions of S-protein RBD samples in HBS-EP+ buffer were injected at a flow rate of 30 μL/min. At the end of the sample injection, the same buffer was allowed to flow over the sensor surface to facilitate dissociation. After a 3 min dissociation time, the sensor surface was regenerated by injecting with 30 of 2M NaCl. The response was monitored as a function of time (sensorgram) at 25° C.

Evaluation of the Inhibition Activity of Sulfated Glycans on Heparin-S-Protein RBD Using Solution Competition SPR. For competition studies between surface heparin and different soluble sulfated glycans and highly negative compounds using SPR, S-protein RBD (250 nM) samples mixed with different concentrations of sulfated glycans in HBS-EP+ buffer were injected over the heparin chip at a flow rate of 30 μL/min, respectively. After each run, dissociation and regeneration were performed. For each set of competition experiments on SPR, a control experiment (only protein) was performed to ensure that the surface was completely regenerated and that the results obtained between runs were comparable. Once the active binding sites on S-protein molecules were occupied by sulfated glycan in the solution, the binding of S-protein to the surface-immobilized heparin was decreased, resulting in a reduction in signal. The same protein samples were also mixed with heparin in BB S-EP+ buffer and were tested to serve as a positive control.

S-protein RBD (250 nM) samples premixed with different concentrations of sulfated glycan (in ½ serial dilutions with HBS-EP+ buffer) were injected over the heparin chip to measure IC₅₀. The IC₅₀ values were calculated from the plots (S-protein binding signal (normalized) versus sulfated glycan concentration in solution).

SPR Solution Competition Study of the Inhibition Sulfated Glycans on the Interaction of Heparin and SARS-CoV-2 Pseudoviral Particles. SARS-CoV-2 pseudotyped viral particles premixed with sulfated glycans in BB S-EP+ buffer were injected over the heparin chip at a flow rate of 30 μL/min. Similarly, when the active binding sites on the pseudotyped viral particles were occupied by sulfated glycans in solution, the binding of the viral particles to the surface-immobilized heparin decreased, resulting in a reduction in signal in RU.

In Vitro SARS-CoV-2 Pseudotyped Virus Neutralization Assay. ACE2 stable cell line generation: Lentiviral particles containing the ACE2-Puro construct were produced by transfecting 12.3 μg psPAX2 (Addgene #12260), 2.5 μg pMD2g (#12259), and 14.7 μg pLenti-hACE2-Puro into HEK293T cells using Lipofectamine 2000 according to the manufacturer's instructions. The plasmids, psPAX2 and pMD2g, were provided by Didier Trono (École Polytechnique Fédérale de Lausanne, Switzerland). A medium exchange was carried out 24 hours after transfection, and 5 mM sodium butyrate (Millipore Sigma, Burlington, MA, USA) was added to the cells in fresh medium. The supernatants from HEK293T cells carrying the lentiviral particles were harvested at 48 and 72 hours. The supernatants were pooled and concentrated using Lenti-X-Concentrator (Takara Bio, Shiga, Japan) according to the manufacturer's instructions. The concentrated lentiviral particles carrying ACE2-Puro were delivered to HEK293T cells in 6-well tissue culture-treated plates. After 48 hours, 4 μg/mL of puromycin was added to Dulbecco's Modified Eagle Medium (DMEM)+10% fetal bovine serum (FBS), and a medium exchange was carried out. The cells were passaged to a T-25 flask and maintained in selection pressure (4 μg/mL puromycin) to remove cells lacking the ACE2-Puro construct.

Production of Spike Pseudotyped Viral Particles. HEK293T cells were seeded in two T175 flasks and cultured in DMEM+10% FBS. At 70-80% confluence, the cells were transfected using Lipofectamine 2000. For production of WT and Delta spike pseudotyped particles, the cells were transfected with 26 μg of psPAX2, 26 μg of pLV-enhanced green fluorescent protein (EGFP) (a gift from Pantelis Tsoulfas (University of Miami, Florida), Addgene plasmid #36083), and 8.7 μg of pHDM-SARS-CoV2-S (BEI Resources #NR52514) per flask. A medium exchange was performed 24 hours after transfection with the addition of 5 mM sodium butyrate (Millipore Sigma, Burlington, MA). The harvest supernatant was collected at 48 and 72 hours and concentrated using Lenti-X-Concentrator according to the manufacturer's instructions. The resuspended viral samples were stored at −80° C. until use.

SARS-CoV-2 Pseudotyped Virus Neutralization Assay: Six different concentrations of sulfated glycans were prepared at a 10-fold serial dilution from 1000 to 0.01 μg/mL in DMEM+1% PenStrep and no FBS. Viral samples were then added at 1:10 dilution, and the mixtures were incubated at 37° C. for 1 hour. The samples were then added to HEK293T-ACE2 cells, plated in 96-well plates at 15,000 cells/well, and incubated for 4 hours at 37° C. Afterwards, a media change was performed with DMEM+1% PenStrep+10% FBS. The cells were cultured for an additional 48 hours and were then stained with 5 μg/mL of Hoechst 33342 and imaged using Cellomics Arrayscan XTI. The infection efficiency was then calculated using the Target Activation Bioapplication. Without wishing to be bound by theory, the results of the experiment represent the percentage (%) of maximum infectivity that could be obtained for the experiment. This was carried out by normalizing the percentage (%) infected value for each sample by the percentage (%) infected value at the 1:10 dilution and 0 μg/mL of compound.

Methods and systems of the present disclosure are advantageous to provide compositions, including sulfated glycans and additional highly negatively charged compounds, with anti-SARS-CoV-2 activity. As discussed above, a small library of sulfated glycans and highly negative compounds including PPS, MPS, etc., was assembled and demonstrated binding to the wild-type and variant SARS-CoV-2 S-proteins and inhibition of viral infectivity in vitro.

In a competition SPR assay, PPS and MPS in solution showed remarkable inhibition activity against chip-surface heparin binding with the wild-type S-protein RBD (with a measured ˜35 nM and ˜9 nM, respectively), which was much lower than the IC₅₀ for heparin (IC₅₀=56 nM). Compositions according to embodiments of the present disclosure, such as those including concentrations of PPS and MPS, display a higher capacity to bind S-protein RBD from VOC, and the pseudotyped viral particles of wild-type/Delta variant, compared with that of heparin alone. The neutralizing effect of PPS and MPS on SARS-CoV-2 pseudotyped virus was confirmed in vitro; the IC₅₀ values for PPS inhibition of heparin binding to pseudotyped virus of WT and Delta variant were 0.45 and 0.07 μg/mL, respectively, and the IC₅₀ values of for MPS were 0.42 and 0.28 μg/mL, respectively. Compositions consistent with the embodiments of the present disclosure can be utilized as effective therapeutic antiviral drugs, particularly against SARS-CoV-2. Further, nasal administration of the compositions according to embodiments of the present disclosure enable simplified treatment, both in patients susceptible to SARS-CoV-2 in a preventative capacity preventative and in those patients with active SARS-CoV-2 infections.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A composition for inhibiting SARS-CoV-2, the composition comprising:
one or more inhibitors configured to bind to spike protein (S-protein) of severe acute respiratory syndrome coronavirus 2 (SARS-CoV-2),
wherein the one or more inhibitors include pentosan polysulfate, mucopolysaccharide polysulfate, sulfated lactobionic acid, sulodexide, a defibrotide, 4-t-butylcalix[X] arene-p-sulfonic acid, or combinations thereof,
wherein X is 6 or 8.

2. The composition according to claim 1, wherein the composition is formulated for nasal delivery.

3. The composition according to claim 1, wherein the 4-t-butylcalix[X] arene-p-sulfonic acid includes GL-2179, GL-2021, GL-2029, GL-288-Y-1, GL-522-Y-1, GL-522Y-1 Calcium, or combinations thereof.

4. The composition according to claim 3, wherein the one or more inhibitors include pentosan polysulfate, mucopolysaccharide polysulfate, or combinations thereof.

5. The composition according to claim 3, wherein the one or more inhibitors have a molecular weight greater than about 5 kDa.

6. The composition according to claim 5, wherein the one or more inhibitors have a molecular weight greater than about 10 kDa.

7. The composition according to claim 1, wherein the composition includes:
one or more additional active ingredients; or
pharmaceutically acceptable adjuvants, diluents, excipients, carriers, or combinations thereof.

8. A method of treating an individual with SARS-CoV-2, comprising:
identifying a SARS-CoV-2 infection in a patient; and
nasally administering an effective amount of a composition to the patient, the composition including one or more inhibitors configured to bind to spike protein (S-protein) of SARS-CoV-2,
wherein the one or more inhibitors include pentosan polysulfate, mucopolysaccharide polysulfate, sulfated lactobionic acid, sulodexide, a defibrotide, GL-2179, GL-2021, GL-2029, GL-288-Y-1, GL-522-Y-1, GL-522Y-1 Calcium, or combinations thereof,
wherein the composition is formulated for nasal delivery.

9. The method according to claim 8, wherein the one or more inhibitors include pentosan polysulfate, mucopolysaccharide polysulfate, or combinations thereof.

10. The method according to claim 8, wherein the one or more inhibitors have a molecular weight greater than about 5 kDa.

11. The method according to claim 10, wherein the one or more inhibitors have a molecular weight greater than about 10 kDa.

12. The method according to claim 8, wherein the composition includes:
one or more additional active ingredients; or
pharmaceutically acceptable adjuvants, diluents, excipients, carriers, or combinations thereof.

13. The method according to claim 8, wherein nasally administering to the patient the effective amount of the composition produces a peak plasma concentration in the patient less than about 0.5 ug/mL.

14. A method of treating or preventing an infection caused by SARS-CoV-2 in a patient, comprising:
administering an effective amount of a composition to a patient susceptible to infection by SARS-CoV-2 to treat or prevent SARS-CoV-2 infection, the composition including one or more inhibitors configured to bind to spike protein (S-protein) of SARS-CoV-2
wherein the one or more inhibitors include one or more sulfated glycans having a plurality of repeating disaccharide subunits,
wherein the disaccharide subunits include 3 or more sulfo groups.

15. The method according to claim 14, wherein administering to the patient the effective amount of the composition includes:
administering the composition nasally, wherein the composition is formulated for nasal delivery.

16. The method according to claim 14, wherein the sulfated glycans include pentosan polysulfate, mucopolysaccharide polysulfate, or combinations thereof.

17. The method according to claim 16, wherein the sulfated glycans have a molecular weight greater than about 5 kDa.

18. The method according to claim 17, wherein the sulfated glycans have a molecular weight greater than about 10 kDa.

19. The method according to claim 14, wherein the composition includes:
one or more additional active ingredients; or
pharmaceutically acceptable adjuvants, diluents, excipients, carriers, or combinations thereof.

20. The method according to claim 14, wherein administering to the patient the effective amount of the composition produces a peak plasma concentration in the patient less than about 0.5 ug/mL.
